(19) United States
(12) Patent Application Publication
(10) Pub. No.: US 2026/0240677 A1
(43) Pub. Date: Aug. 20, 2026

(54) Flavored Tooth Guard Device

(71) Applicant: Krystal Smith, Murfreesboro, TN (US)

(72) Inventor: Krystal Smith, Murfreesboro, TN (US)

(21) Appl. No.: 19/053,530

(22) Filed: Feb. 14, 2025

(51) Int. Cl. A61C 19/06

(52) U.S. Cl. CPC A61F 5/566

(57) ABSTRACT

A flavored tooth guard device for dulling a row of teeth includes a strip that is releasably secured to a row of teeth of a user wherein the strip dulls the row of teeth. An adhesive material is positioned on the strip to adhesively secure the strip to the row of teeth. A gelatin is positioned within the strip. The strip is biodegradable wherein the strip degrades when each strip of the pair of strips is exposed to friction between the row of teeth and an object. The gelatin is released from the strip once the strip is degraded.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to tooth strips and more particularly pertains to a new tooth strip for dulling a row of teeth.

### (2) Description of Related Art including information disclosed under 37 CFR 1.97 and 1.98.

The prior art relates to tooth strips. Tooth strips are most commonly used to whiten teeth and are commonly referred to as tooth whitening strips. Some mouth guards have been designed to protect teeth from damage caused by tooth grinding, or to inhibit injury when a user is playing sports. Retainers have also been designed to help inhibit rows of teeth from shifting, or to help straighten rows of teeth. However, the prior art lacks disclosure of a tooth strip which can be used to dull the teeth to inhibit the teeth from scratching or biting another user, and which can release a flavored gelatin into the mouth of the user.

### (h) BRIEF SUMMARY OF THE INVENTION

An embodiment of the disclosure meets the needs presented above by generally comprising a strip that is configured to be releasably secured to a row of teeth of a user wherein the strip is configured to dull the row of teeth. An adhesive material is positioned on the strip wherein the adhesive material is configured to adhesively secure the strip to the row of teeth. A gelatin is positioned within the strip. The strip is biodegradable wherein the strip is configured to degrade when each strip of the pair of strips is exposed to friction between the row of teeth and an object. The gelatin is released from the strip once the strip is degraded.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a flavored tooth guard device according to an embodiment of the disclosure.

FIG. 2 is a rear isometric view of an embodiment of the disclosure.

FIG. 3 is a cross-sectional view of an embodiment of the disclosure.

FIG. 4 is an in-use view of an embodiment of the disclosure.

FIG. 5 is an in-use view of an embodiment of the disclosure.

### (j) DETAILED DESCRIPTION OF THE INVENTION

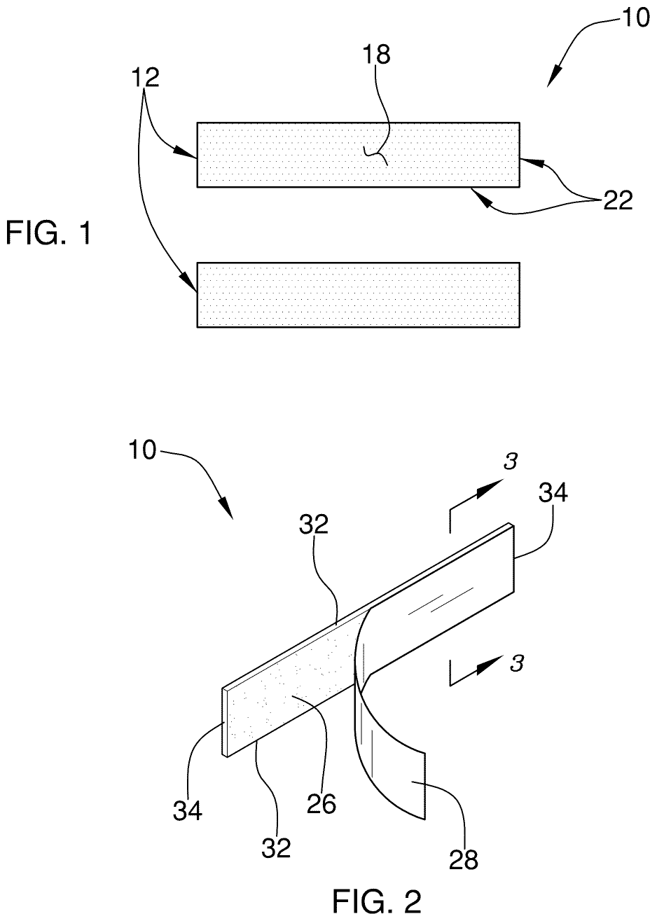

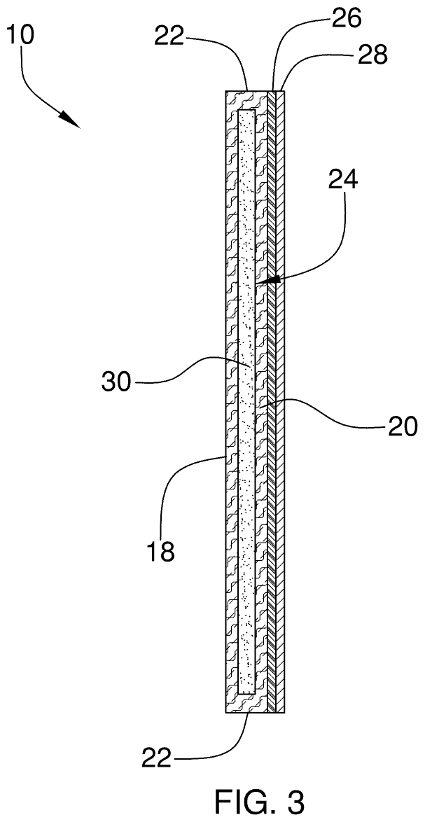

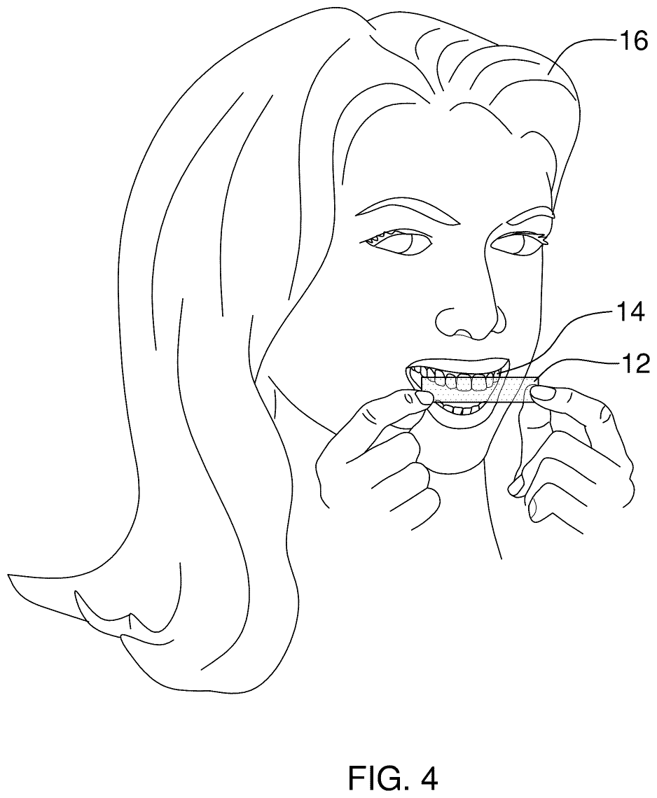

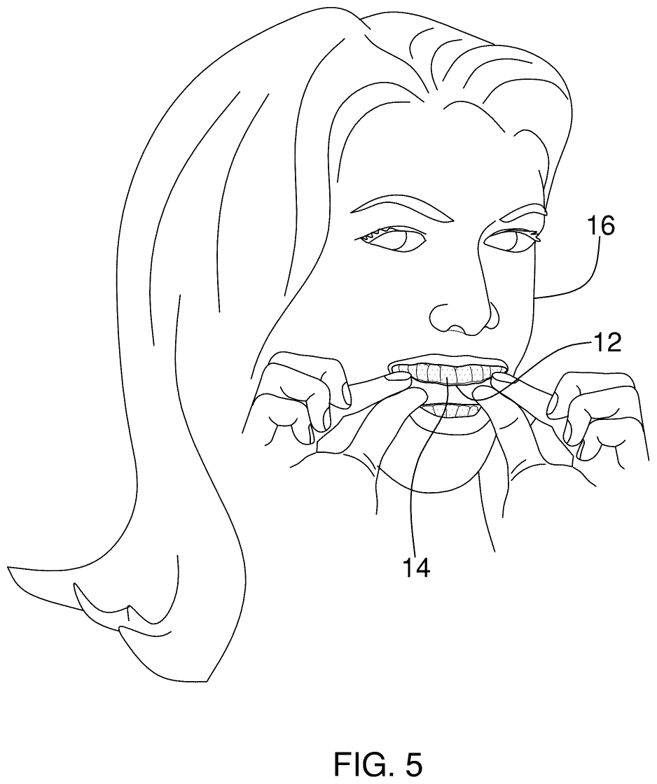

With reference now to the drawings, and in particular to FIGS. 1 through 5 thereof, a new tooth strip embodying the principles and concepts of an embodiment of the disclosure and generally designated by the reference numeral 10 will be described.

As best illustrated in FIGS. 1 through 5, the flavored tooth guard device 10 generally comprises a strip 12 that is configured to be releasably secured to a respective row of teeth 14 of a user 16. In some embodiments, the strip 12 may be one of a pair of strips 12. In such embodiments, each strip of the pair of strips 12 is configured to be releasably secured to the respective row of teeth 14 of the user 16, each strip of the pair of strips 12 being configured to cover a crown of each tooth of the respective row of teeth 14 wherein each strip of the pair of strips 12 is configured to dull the respective row of teeth 14. More specifically, the pair of strips 12 are configured for positioning over all thirty-two teeth within a mouth of the user 16 to dull all thirty-two teeth and inhibit all of the teeth from scratching or biting an object being positioned within a mouth of the user 16. Each strip 12 is generally configured for positioning over sixteen of the thirty-two teeth of the user 16.

Each strip of the pair of strips 12 generally includes a front surface 18. The front surface 18 is typically rectangular. The front surface 18 may be planar. A rear surface 20 of each strip of the pair of strips 12 is also typically rectangular. The rear surface 20 may be planar because the rear surface 20 is generally configured to be positionable against the respective row of teeth 14 of the user 16. A peripheral edge 22 is generally coupled to and extends between the front surface 18 and the rear surface 20. The peripheral edge 22 may have a pair of elongated sides 33 and a pair of shortened sides 34 extending between the pair of elongated sides 33 wherein each strip of the pair of strips 12 is rectangular. An interior space 24 is generally defined by the front surface 18, the rear surface 20, and the peripheral edge 22.

An adhesive material 26 is positioned on each strip of the pair of strips 12. The adhesive material 26 is generally configured to adhesively secure each strip of the pair of strips 12 to the respective row of teeth 14 of the user 16. The adhesive material 26 is generally positioned on the rear surface 20 of each strip of the pair of strips. For example, the adhesive material 26 may be positioned to cover the rear surface 20 of each strip of the pair of strips 12.

A cover 28 may be removably affixed to the adhesive material 26. The cover 28 is generally configured to inhibit debris from damaging the adhesive material 26 while the cover 28 is affixed to the adhesive material 26. The cover 28 may have a size that is complementary to a size of the rear surface 20 of each strip of the pair of strips 12. More generally, the cover 28 is positioned to cover the adhesive material 26 while the cover 28 is affixed to the adhesive material 26.

A gelatin 30 is generally positioned within the interior space 24 of each strip of the pair of strips 12. The gelatin 30 is typically made from edible materials wherein the gelatin 30 is configured for consumption by the user 16. For example, the gelatin 30 may be made from processed collagen extracted from animals and may also be made from vegetable gums and seaweed extracts, although other edible materials are also contemplated.

The gelatin 30 may have a flavor that is configured to facilitate consumption of the gelatin 30 by the user 16. In other words, the gelatin 30 may be flavored to enhance the experience for the user 16 as the gelatin 30 is consumed. For example, the flavor may be grape, raspberry, blueberry, orange, lemon, or any other flavor which the user 16 enjoys tasting. In some embodiments, each strip of the pair of strips 12 may have a respective flavor such that one strip of the pair of strips has a first flavor and the other strip has a second flavor.

The pair of strips 12 are generally biodegradable. More specifically, at least the front surface 18 of each strip of the pair of strips 12 may be biodegradable wherein the front surface 18 of each strip of the pair of strips 12 is configured to degrade when the front surface 18 of each strip of the pair of strips 12 is exposed to friction between the respective row of teeth 14 and an object. In alternative embodiments, the rear surface 20 or the peripheral edge 22 may also each be biodegradable. For example, the object may be positioned within the mouth of the user 16. Friction may be created as the object is rubbed against the front surface 18 of the pair of strips 12. In a more specific example, the user 16 may be performing fellatio with a partner, in which case the object may be a penis of the partner. The user 16 may secure the pair of strips 12 to each respective row of teeth 14 to inhibit each respective row of teeth 14 from scratching or biting the penis of the partner. As each respective row of teeth 14 rubs against the penis, friction between each strip of the pair of strips 12 and the penis causes the pair of strips 12 to degrade.

The gelatin 30 is released from the interior space 24 of each strip of the pair of strips 12 when the front surface 18 of each strip of the pair of strips 12 is degraded. For example, the front surface 18 of each strip of the pair of strips 12 may be made from polysaccharides such as cellulose, chitin, and pullulan, although other materials which degrade when exposed to frictional forces and which are safe for the user 16 to ingest are also contemplated. In other embodiments, the rear surface 20 and the peripheral edge 22 of each strip of the pair of strips 12 may also degrade when each strip of the pair of strips 12 is exposed to friction as described above.

In use, the pair of strips 12 can protect sensitive skin of the partner of the user 16 from being scratched, bitten, or otherwise injured while the user 16 is wearing the pair of strips 12. The user 16 may use both strips of the pair of strips 12 to cover both rows of teeth a single strip 12. Alternatively, the user 16 can use one strip 12 to cover a single row of teeth 14. Once the pair of strips 12 degrade, the gelatin 30 is released into the mouth of the user 16. Said gelatin 30 may be flavored to enhance a taste of the gelatin 30 for the user 16. Thus, the experience for both the user 16 and the partner can be enhanced.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A mouth guard assembly comprising:
a strip being configured to be releasably secured to a row of teeth of a user wherein the strip is configured to dull the row of teeth;
an adhesive material being positioned on the strip wherein the adhesive material is configured to adhesively secure the strip to the row of teeth; and
a gelatin being positioned within the strip, the strip being biodegradable wherein the strip is configured to degrade when each strip of the pair of strips is exposed to friction between the row of teeth and an object, the gelatin being released from the strip once the strip is degraded.

2. The mouth guard assembly of claim 1, wherein the strip is one of a pair of strips, each strip of the pair of strips being configured to be releasably secured to a respective row of teeth of the user.

3. The mouth guard assembly of claim 1, wherein the strip is configured to cover a crown of each tooth of the row of teeth wherein the strip is configured to dull the respective row of teeth.

4. The mouth guard assembly of claim 1, the strip further comprising:
a front surface;
a rear surface;
a peripheral edge being coupled to and extending between the front surface and the rear surface; and
an interior space being defined by the front surface, the rear surface, and the peripheral edge.

5. The mouth guard assembly of claim 4, wherein the gelatin is positioned within the interior space of the strip.

6. The mouth guard assembly of claim 1, wherein the gelatin is made from edible materials wherein the gelatin is configured for consumption by the user.

7. The mouth guard assembly of claim 1, wherein the gelatin has a flavor being configured to facilitate consumption of the gelatin by the user.

8. A mouth guard assembly comprising:
a pair of strips, each strip of the pair of strips being configured to be releasably secured to a respective row of teeth of a user, each strip of the pair of strips being configured to cover a crown of each tooth of the respective row of teeth wherein each strip of the pair of strips is configured to dull the respective row of teeth, each strip of the pair of strips including:
a front surface being rectangular, the front surface being planar;
a rear surface being rectangular, the rear surface being planar;
a peripheral edge being coupled to and extending between the front surface and the rear surface, the peripheral edge having a pair of elongated sides and a pair of shortened sides extending between the pair of elongated sides wherein each strip of the pair of strips is rectangular; and
an interior space being defined by the front surface, the rear surface, and the peripheral edge;
an adhesive material being positioned on each strip of the pair of strips, the adhesive material being configured to adhesively secure each strip of the pair of strips to the respective row of teeth of the user, the adhesive material being positioned to cover the rear surface of each strip of the pair of strips;
a cover being removably affixed to the adhesive material wherein the cover is configured to inhibit debris from damaging the adhesive material while the cover is affixed to the adhesive material, the cover having a size being complementary to a size of the rear surface of each strip of the pair of strips wherein the cover is positioned to cover the adhesive material while the cover is affixed to the adhesive material; and
a gelatin being positioned within the interior space of each strip of the pair of strips, the gelatin being made from edible materials wherein the gelatin is configured for consumption by the user, the gelatin having a flavor being configured to facilitate consumption of the gelatin by the user, the front surface of each strip of the pair of strips being biodegradable wherein the front surface of each strip of the pair of strips is configured to degrade when the front surface of each strip of the pair of strips is exposed to friction between the respective row of teeth and an object, the gelatin being released from the interior space of each strip of the pair of strips while the front surface of each strip of the pair of strips is being degraded.
